(19) United States
(12) Patent Application Publication
(10) Pub. No.: US 2026/0249856 A1
(43) Pub. Date: Aug. 27, 2026

(54) Driver Intoxication Measurement From Passive Face and Voice Analysis

(71) Applicant: Blueskeye AI Ltd, Nottingham (GB)

(72) Inventors: Michel François Valstar, Nottingham (GB); Mani Kumar Tellamekala, Birmingham (GB); Anthony Brown, Nottingham (GB); Adrian Cornelius Marinescu, Nottingham (GB); Thomas Smith, Sheffield (GB); Shashank Jaiswal, Nottingham (GB)

(21) Appl. No.: 19/544,438

(22) Filed: Feb. 19, 2026

Related U.S. Application Data
(60) Provisional application No. 63/762,021, filed on Feb. 23, 2025.
(60) Provisional application No. 63/976,763, filed on Feb. 5, 2026.

(51) Int. Cl. B60W 40/08; B60K 28/06

(52) U.S. Cl. CPC B60W 40/08; B60K 28/06; B60W 2040/0836

(57) ABSTRACT

Described is reducing the number of instances when a potential driver needs to use a breathalyzers via analysis of the driver's passive expressive gait, face and voice analysis. Fitness to drive is tested both before setting off and continue measuring during a drive. Reliable indicators for intoxication can be collected passively from visual behavior as a driver approaches (using the cars external cameras) and/or enters the car (using DMS cameras); and from face and voice behavior if the driver interacts with a voice assistant and as part of that utters a short, known sentence.

### PRIOR APPLICATIONS

This application claims the benefit of the following two applications, which are incorporated by reference in their entirety:
1. U.S. Provisional Patent Application No. 63/762,021, filed on Feb. 23, 2025; and
2. U.S. Provisional Patent Application No. 63/976,763, filed on Feb. 5, 2026.

### FIELD OF THE DISCLOSURE

The present disclosure relates generally to improved techniques for measurement of driver intoxication based on passive face and voice analysis.

### BACKGROUND

Alcohol interlocks are already in use in some countries. These have been used widely in North America and Sweden in rehabilitation schemes for repeat offenders driving with a blood alcohol content over the legal limit. They are also used in government and company fleet cars in Sweden. Trials have been taking place in various countries such as the US, Australia, Canada, Belgium and Sweden. However, they involve the use of a breathalyzer which is cumbersome and time consuming.

As described by the European Commission, alcohol interlocks are automatic control systems which are designed to prevent driving with excess alcohol by requiring the driver to blow into an in-car breathalyzer before starting the ignition. The alcohol interlock can be set at different levels and limits.

### What Road Safety Problem do Alcohol Interlocks Address:

Excess alcohol contributes to about 25% of all road deaths in Europe. A large part of the problem consists of ‘high risk offenders’ who offend regularly and/or exceed legal blood alcohol levels by a large amount. With a BAC of 1.5 g/l the crash rate for fatal crashes is about 200 times that of sober drivers. In some countries e.g. Britain, levels of police enforcement of legal limits has dropped in recent years, leading to increases in drinking and driving. Alcohol interlocks address excess alcohol in the driving population at large, as well as repeat offenders.

### How Effective:

Large scale quantitative research on alcohol ignition interlocks in use has shown that alcohol interlocks are 40 to 95 percent more effective in preventing drink driving recidivism than traditional measures such as license withdrawal or fines. A literature review (UK Department for Transport, 2004) showed a recidivism reduction of about 28-65% in the period where the alcohol interlock is installed compared with the control groups who were not using the alcohol interlock. An EU study indicated that alcohol interlocks need to be fitted permanently to have an effect, since after removal of the lock recidivism increases again. Alcohol interlocks clearly have an important role to play within rehabilitation programs.

There has been no evaluation of the impact that alcohol interlocks used in commercial transport have on road safety but Swedish companies report that fitting alcohol interlocks prevented excess alcohol amongst fleet drivers.

### Benefits to Cost:

In a recent cost benefit analysis, estimations are made for implementing alcohol interlocks for drivers caught twice with a BAC between 0.5 g/l and 1.3 g/l and for drivers caught with a BAC above 1.3 g/l in several countries.

### Benefits to Cost of Alcohol Interlocks in Different Countries:

For the Netherlands, the reduction of 35 traffic fatalities annually is valued at 4.8 million per death, leading to a benefit of 168 million Euros. Benefit/cost ratio=4.1.

For the Czech Republic, the 8 fatalities prevented are counted at 1.1 million Euro/death, leading to estimated benefits of 9 million Euro/year. Benefit/cost ratio=1.6.

For Norway, the benefits are calculated as 5.5 deaths less per year a rate of 5.9 million Euro per death, or at 32.5 million Euro/year. Benefit/cost ratio=4.5.

For Spain, the reduction with 86.5 deaths/year at 800.000 Euro per death would imply benefits of 69 million Euro/year. Benefit/cost ratio=0.7.

### Who Uses Alcohol Interlocks Now:

These have been used widely in North America and Sweden in rehabilitation schemes for repeat offenders driving with a blood alcohol content over the legal limit. They are also used in government and company fleet cars in Sweden. Trials have been taking place in various countries such as the US, Australia, Canada, Belgium and Sweden.

### Alcohol Interlock Policy and Practice in Sweden:

In a trial running from 1999 to 2002 in Sweden, 300 alcohol interlocks were installed in commercial passenger and goods transport. Subsequently, manufacturers such as Volvo and Toyota offer installation of alcohol interlocks in trucks as a dealership option in Sweden. One transport company in Sweden decided to equip all their 4000 vehicles with alcohol interlock systems before the end of 2006. From 2006 all trucks of 3.5 tons and over, which are contracted by the Swedish Road Administration (SRA) for more than 100 hours per year, have to be fitted with alcohol interlocks (SRA, ITS Strategy, 2006-9).

More than 5000 company cars in Sweden are today equipped with alcohol interlocks and the number is growing rapidly. The Swedish Driving Schools Association has fitted all their 800 vehicles with alcohol interlocks. In 2007 Volvo launched an alcohol interlock for normal use in cars.

In 2004 the SRA required that all the SRA's purchased or leased vehicles must be equipped with alcohol ignition interlocks during 2008 at the latest. By 2010, 50% of all new cars used by companies in Sweden should have alcohol interlocks.

### Next Steps for Implementation:

Sweden has recently introduced a strategy on alcohol interlocks and the rehabilitation of offenders and has proposed that from the year 2012, all new cars should have an alcohol ignition interlock installed. However, Sweden is the only EU Member State that uses alcohol locks at present, even in rehabilitation programs, although experiments are being carried out in Spain, Belgium, Germany, and Norway. More widespread application will require a technical specification to be devised for alcohol interlocks as well as debate about their use, whether for rehabilitation or in normal use.

Breathalyzers measuring blood alcohol volume are accurate and well-established but are cumbersome and time-consuming to use, and would be a serious disruption if they had to be done every time to start the car.

Accordingly, reducing the number of instances when a person would need to use the breathalyzers using passive expressive gait, face and voice analysis would be beneficial.

### SUMMARY

Measuring fitness to drive from gait, face and voice behavior takes some time. To be non-disruptive it should be passive, i.e. not involve any explicit test steps that appear unnecessary for the occupant to get on their way. On the other hand, the highest signal to noise ratio of intoxication from expressive behavior is achieved when occupants undergo a specific, known, well-validated test. It is non-trivial to design a test that is short, not disruptive, and captures the strongest face and voice signal.

Reliable indicators for intoxication can be collected passively prior to setting off:
From visual behavior as a driver approaches (using the cars external cameras) and/or enters the car (using Driver Monitoring System cameras); and
From face and voice behavior if someone interacts with a voice assistant and as part of that utters a short, known sentence.

By combining these techniques, a measure of intoxication can be derived in a very short time prior to setting off.

It is important from a safety perspective to test fitness to drive both before setting off and continue measuring during a drive. Measuring only before setting off is insufficient as it may take time for a substance used by a driver to take effect, and the driver may consume substances after the engine has started.

Reliable indicators for intoxication can be collected passively whilst a person is driving:
From face and voice behavior, where face behavior includes but is not limited to eye movements; and
From driver actions: drinking actions will increase probability of intoxication.

### BRIEF DESCRIPTION OF THE DRAWINGS

The accompanying figures, where like reference numerals refer to identical or functionally similar elements throughout the separate views, together with the detailed description below, are incorporated in and form part of the specification, serve to further illustrate embodiments of concepts that include the claimed invention and explain various principles and advantages of those embodiments.

FIG. 1 shows a flowchart of face and voice recognition to determine if a human can drive a car.

FIG. 2 and FIG. 3 each show data collected from a participant during a trial of visually guided oculomotor tracking.

FIG. 4 shows a plot of participants' BrAC levels.

FIG. 5 shows a plot of Impact Factors on Smooth Pursuit Gaze Angle Pitch (Mean_rmse_pitch) (vertical).

FIG. 6 shows a plot of Smooth Pursuit Gaze Angle Pitch (Mean_rmse_pitch) (vertical) vs. Alcohol Concentration.

FIG. 7 shows a plot of Smooth Pursuit Gaze Angle Pitch (Mean_rmse_pitch) (vertical) vs. Trial Progression.

FIG. 8 shows a plot of Impact Factors on Saccade Mean_peak_speed_deg_per_second.

FIG. 9 shows a plot of Saccade Mean_peak_speed_deg_per_second vs. Alcohol Concentration.

FIG. 10 shows a plot of Impact Factors: Fixation Raw_std_dev_yaw (horizontal).

FIG. 11 shows a plot of Fixation Raw_std_dev_yaw (horizontal) vs. Alcohol Concentration.

FIG. 12 shows a plot of Audio mfcc_mean_20 vs. Alcohol Concentration.

FIG. 13 shows a plot of Audio spectral_rolloff mean vs. Alcohol Concentration.

FIG. 14 shows a plot of Audio spectral_centroid_mean vs. Alcohol Concentration.

FIG. 15 shows a plot of data collected from a participant related to Smooth Pursuit Gaze Angle Pitch (Mean_rmse_pitch) (vertical).

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

The following tasks are considered for evaluation and refinement.

Passive intoxication test 1 begins as the driver approaches the car. Here, the car system analyzes the characteristics of the driver to determine if the driver may be impaired. Such characteristics may include gait, appearance, eye movement features, head movement features, and the like.

Passive intoxication test 2 begins with the voice assistant in the car challenges the human in the driver seat. Starting the voice assistant also starts the sensors that measure the driver's expressive behavior, which must include in particular a near infrared (NIR) camera and the microphone, and may include sensors in the seat, steering wheel, or elsewhere, and may include wireless connection of a wearable device to the intoxication analysis system in the car. The ensures that a known sentence is always captured before the car sets off. The driver starts the car engine with a voice command, e.g. ‘Hello, start the car’. The command can be varied in the way it's delivered e.g. ‘Hi, start the car’, ‘Eyup, start the engine’ or any variation of this command that the automatic speech recognition system will interpret as an instruction to start the engine. After starting the engine, but before enabling the driver to drive off, the car's voice assistant will ask using natural language whether the driver intends to drive themselves or whether the car should drive autonomously for the occupant. An example question from the voice assistant could be: ‘Would you like to drive, or should I?’. The drive would respond with their preference, allowing variations in their response in the same way they instructed the car to turn on the engine.

Based on this interaction, the fitness to drive analysis estimates intoxication levels and determines whether the driver is allowed to take control of the vehicle.

The voice assistant may ask more optional questions if appropriate, e.g. about routing, music or other things. Answers to this may be used to get a more confident or more accurate reading of intoxication. The system may ask follow up questions if it is uncertain about the intoxication level. The system may have a limit to the time that the passive intoxication test 2 takes. The system measures confidence in its measurement. If the confidence is too low, it will delegate the decision to a more accurate system such as a breathalyzer. If the passive system detects intoxication prior to setting off, it may require the human to do a breathalyzer, or may deny the human the ability to drive directly. The decision to do a breathalyzer test as a second test may depend on the probability of intoxication and the confidence level of the passive tests. High probability and confidence may be sufficient to immediately deny the occupant in the driver seat to take control of the vehicle.

This is sufficiently accurate to avoid doing a breathalyzer test to determine alcohol intoxication in the vast majority of cases prior to setting off. Other aspects of fitness to drive may be measured as well, including intoxication by other drugs, and fatigue.

The use as test input features include: gait, tone of voice, slurring, etc., plus pupil dilations, facial muscle actions including eye gaze patterns and blinking, head actions and other face and voice behavior to predict fitness to drive. All test input features will be analyzed for their temporal dynamics.

Face and voice recognition could be used for enhanced security, and for improved fitness to drive measurement by comparing a person's behavior to their baseline profile.

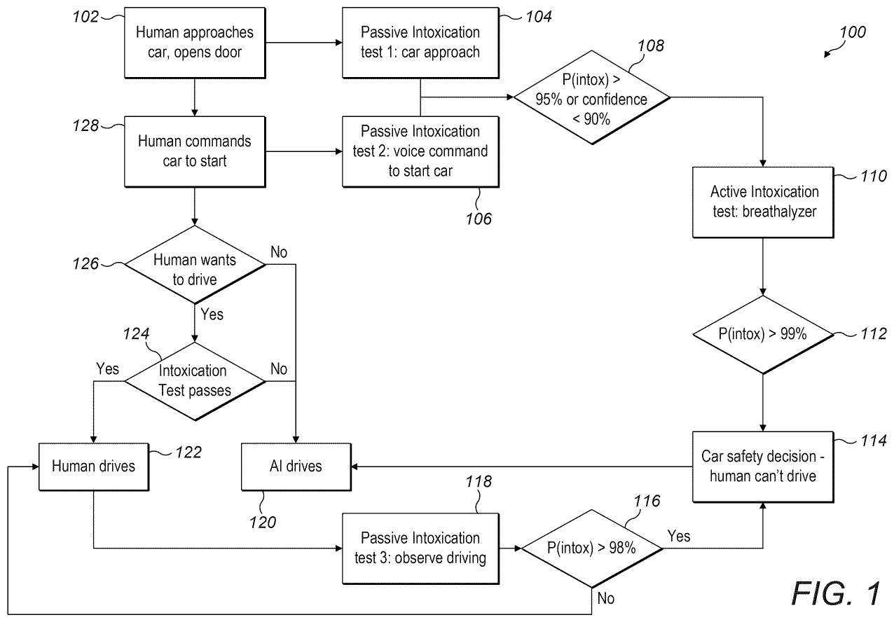

A diagram of how the system works is shown in FIG. 1.

FIG. 1 shows a flowchart 100 where the system begins passive intoxication test 1 with a human approaching the car and opens the door 102. The system performs a passive intoxication test 1 upon car approach 104. Next, the human verbally commands the car to start 128. The system then performs a passive intoxication test 2 upon voice command to start the car 106.

Based on the results of passive intoxication tests, the system determines the probability of whether the driver is intoxicated 108. If the probability is greater than 95% or a confidence of less than 90%, the system activates an active intoxication test via breathalyzer 110. The system determines if the probability of intoxication is greater than 99% 112. If yes, the system determines that the human cannot drive 114 and activates AI driving the car 120.

Meanwhile, after the human verbally commands the car to start 128, the system queries the driver if he or she wants to drive 126. If no, the AI drives the car 120. If yes, the system determines if the intoxication tests have been passed 124. If no, the AI drives the car 120. If yes, the human drives the car 122.

While the human drivers the car 122, the system continues to perform passive intoxication tests 3 118. The system determines if the probability of intoxication is greater than 98% 116. If yes, the car safety decision is made that the human can't drive 114 and the AI drives the car 120. If no, the human continues to drive the car 122.

Note that the threshold values for deciding when a breathalyzer test is necessary or when the driver cannot drive are example values and exact values will depend on regulation and implementation considerations.

Not all test input features will be used for all tests.

The passive intoxication test 1 at the human's approach to the car may be optional. The advantages include:
Reduces the number of times a person would have to use a breathalyzer, thereby reducing the time required to set off with their car whilst maintaining almost the same level of driver and occupant safety.
Improves driver and occupant safety by continuously monitoring driver intoxication and requiring an AI control handover in case a driver becomes intoxicated during the journey.

### II. Intoxication—Oculometric and Voice Measures

### A. Summary

### 1. Overview

A study evaluated the sensitivity of visually guided eye movements and vocal acoustic features as biomarkers for sub-legal alcohol intoxication. Utilizing Linear Mixed-Effects Models across participants, the Smooth Pursuit Gaze Angle Pitch (Mean_rmse_pitch) (vertical) is a highly significant indicator of oculomotor change (p=0.001), independent of subjective fatigue. Conversely, horizontal metrics proved robust, demonstrating that vertical gaze control is more susceptible to low-level impairment than horizontal tracking.

The acoustic analysis reveals that Audio mfcc_mean_20, Audio spectral_rolloff_mean, and Audio spectral_centroid_mean serve as elite audio biomarkers (p<0.01).

### 2. Methodology

### a. Statistical Analysis Method

The relationship between physiological biomarkers and intoxication was evaluated using Linear Mixed Effects Models (LMM), estimated via Restricted Maximum Likelihood (REML). This approach was chosen to account for the hierarchical structure of the data, where multiple observations are nested within individual participants.

For every analyzed feature (gaze and voice), a unified regression formula was applied:

$$Feature ∼ BrAC ( Alcohol ) + Trial Number ( Time ) + KSS Score ( Fatigue ) .$$

The Karolinska Sleepiness Scale (KSS) measures the subjective level of sleepiness at a particular time during the day. On this scale, subjects indicate a level which best reflects the psycho-physical state experienced in the last 10 minutes. The KSS is a measure of situational sleepiness.

Scoring of KSS is on a 9 point scale:
1=extremely alert
2=very alert
3=alert
4=rather alert
5=neither alert nor sleepy
6=some signs of sleepiness
7=sleepy, but no effort to keep awake
8=sleepy and some effort to keep awake
9=very sleepy, great effort to keep awake, fighting sleep

Fixed Effects: This allows the model to simultaneously test the impact of alcohol concentration while mathematically “subtracting” the effects of task habituation (Trial Number) and subjective tiredness (KSS).

Random Effects (Group Variance): A random intercept was assigned to each Participant ID. This accounts for the fact that individuals have different “sober baselines.” The Group Variance represents this inter-individual diversity; by including it, the model ensures that the reported p-values for alcohol are not skewed by one person naturally having “shakier” eyes or a “harsher” voice than another.

The p-value indicates the probability that the observed change in a biomarker (the Coefficient) occurred by chance if alcohol actually had no effect. Statistical significance is set at p<0.05. For example, a p-value of 0.001 for Vertical Deviation RMSE indicates a 99.9% confidence that the observed oculomotor degradation is a direct result of alcohol concentration, independent of individual baselines or fatigue levels.

The Residual Scale represents the “unexplained noise” remaining in the signal after accounting for all predictors. The low residual values in some of the models indicate high measurement precision.

### b. Forest Plot Description

A Forest plot presents the impact of factors (e.g.: Alcohol (BrAC), Trial Progression, Fatigue (KSS)) for a given feature. The x-axis represents the Effect size, corresponding to the regression coefficients (reported in the tables in column “Coefficient”) derived by the Linear Mixed-Effects Models. The black dots indicate the point estimate for the coefficient for each predictor. The horizontal whiskers extending from the dot represent the 95% Confidence Intervals (CI) reported in the tables in column “[95% Conf. Interval]”.

The Effect size quantifies the expected change in a feature for every one-unit increase in the predictor (e.g., 1.0 mg/L increase in BrAC or 1 point on the KSS scale).

The vertical dotted line going through the x-axis origin represents the Null Hypothesis. If a predictor's 95% Confidence Interval (the whiskers) does not cross this zero line, the effect is considered statistically significant (p<0.05).

### 3. Glossary of Terms

Table 1A and Table 1B below each describes the features that were used in the analysis, the task they were derived on as well as how they were derived.

TABLE 1A
(Visual):

| | | | How it was |
|---|---|---|---|
| Task | Internal variable name | Descriptive name | derived |
| Smooth Pursuit | Gaze Angle Pitch | Vertical Smooth | Quantifies vertical |
| | (Mean_rmse_pitch) (vertical) | Pursuit RMSE | instability during |
| | | | horizontal |
| | | | tracking. An |
| | | | “intended path” |
| | | | was generated by |
| | | | applying a 2nd- |
| | | | order Savitzky- |
| | | | Golay filter (0.3 s |
| | | | window) to the |
| | | | vertical pitch data |
| | | | (degrees). RMSE |
| | | | was then |
| | | | calculated |
| | | | between the raw |
| | | | pitch signal and |
| | | | this smoothed |
| | | | idealized |
| | | | trajectory. |
| | Gaze Angle Mean Yaw | Horizontal | Measures |
| | (Mean_rmse_yaw) | Smooth Pursuit | horizontal tracking |
| | (horizontal) | RMSE | precision. An |
| | | | “intended path” |
| | | | was generated by |
| | | | applying a 2nd- |
| | | | order Savitzky- |
| | | | Golay filter (0.3 s |
| | | | window) to the |
| | | | raw horizontal |
| | | | yaw data |
| | | | (degrees). RMSE |
| | | | was calculated as |
| | | | the deviation of |
| | | | raw yaw from this |
| | | | smoothed intended |
| | | | path |
| Fixation | Raw_std_dev_yaw | Horizontal | Calculated as the |
| | (horizontal) | Fixation Stability | standard deviation |
| | | | of the horizontal |
| | | | raw yaw angle |
| | | | (degrees) while |
| | | | viewing a static |
| | | | target. The first |
| | | | 0.5 s of data was |
| | | | discarded to |
| | | | eliminate post- |
| | | | saccadic |
| | | | movements. |
| | Mean_std_dev_pitch | Vertical Fixation | Calculated as the |
| | (vertical) | Stability | standard deviation |
| | | | of the vertical |
| | | | smoothed pitch |
| | | | angle (degrees) |
| | | | while viewing a |
| | | | static target. The |
| | | | first 0.5 s of data |
| | | | was discarded to |
| | | | eliminate post- |
| | | | saccadic |
| | | | movements. |
| Saccade | Mean_peak_speed_deg_per_s | Saccadic Peak | The maximum |
| | | Velocity | instantaneous |
| | | | speed calculated |
| | | | across all |
| | | | monotonic |
| | | | segments |
| | | | (minimum 5 |
| | | | frames) within the |
| | | | saccade window. |
| | | | By requiring |
| | | | directional |
| | | | consistency for at |
| | | | least 5 frames, the |
| | | | algorithm ensures |
| | | | the peak speed |
| | | | reflects the high- |
| | | | velocity ballistic |
| | | | phase of the eye |
| | | | movement rather |
| | | | than momentary |
| | | | sensor noise or |
| | | | high-frequency |
| | | | jitter. |

Root Mean Squared Error (RMSE) is a statistical metric that measures the average magnitude of prediction errors in a model, calculated as the square root of the average of squared differences between predicted and actual values. A lower RMSE indicates higher accuracy and better fit, with a 0.0 value representing a perfect model.

TABLE 1B
(Audio):

| Task | Internal variable name | Unit | How it was derived |
|---|---|---|---|
| Speaking | mfcc_mean_20 | dB (Relative) | Calculated using the |
| task | | | librosa.feature.mfcc library. It |
| | | | represents the 20th coefficient |
| | | | of the Mel-frequency cepstrum, |
| | | | capturing fine-grained spectral |
| | | | envelopes through a Discrete |
| | | | Cosine Transform (DCT). |
| | | | The mel-frequency cepstrum |
| | | | (MFC) is a representation of the |
| | | | short-term power spectrum of a |
| | | | sound, based on a linear cosine |
| | | | transform of a log power |
| | | | spectrum on a nonlinear mel |
| | | | scale of frequency. Mel- |
| | | | frequency cepstral coefficients |
| | | | (MFCCs) are coefficients that |
| | | | collectively make up an MFC. |
| | spectral_rolloff_mean | Hz | Derived via |
| | | | librosa.feature.spectral_rolloff. |
| | | | It identifies the frequency below |
| | | | which a specified percentage |
| | | | (such as 95%, 90%, 85%, 75%, |
| | | | or 50%) of the total spectral |
| | | | energy resides. |
| | spectral_centroid_mean | HZ | Calculated using |
| | | | librosa.feature.spectral_centroid. |
| | | | It represents the “center of |
| | | | mass” of the spectrum, weighted |
| | | | by the intensity of each |
| | | | frequency. It is calculated via |
| | | | the amplitude-weighted mean of |
| | | | frequencies present in a sound. |

### B. Experimental Study Design and Protocol

### 1. Task Paradigm

### a. Oculometric Measures

The study employed a visually guided oculomotor tracking paradigm designed to elicit three distinct types of eye movements: (1) Smooth Pursuit; (2) Saccades; and (3) Fixations.

The stimulus was presented on a screen in front of the participant at a distance of about 1 meter. The task sequence was structured to evaluate both the continuous tracking ability and the rapid-response characteristics of the participant's oculomotor system.

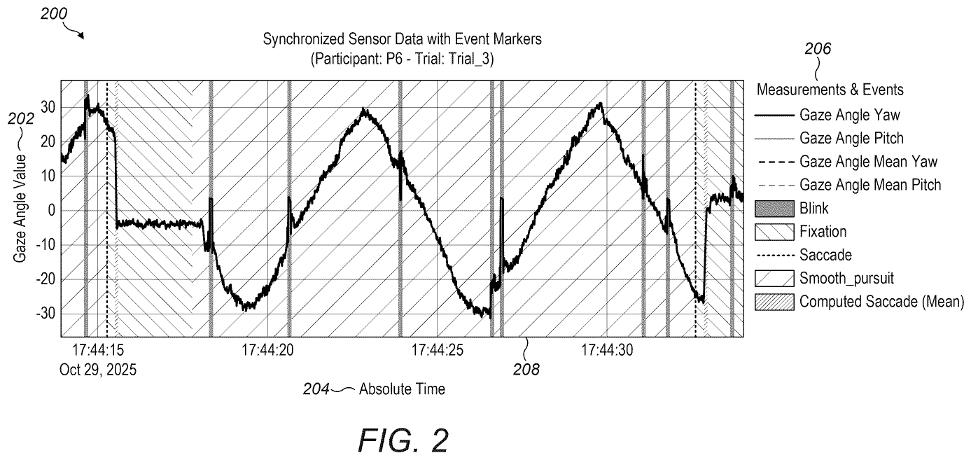

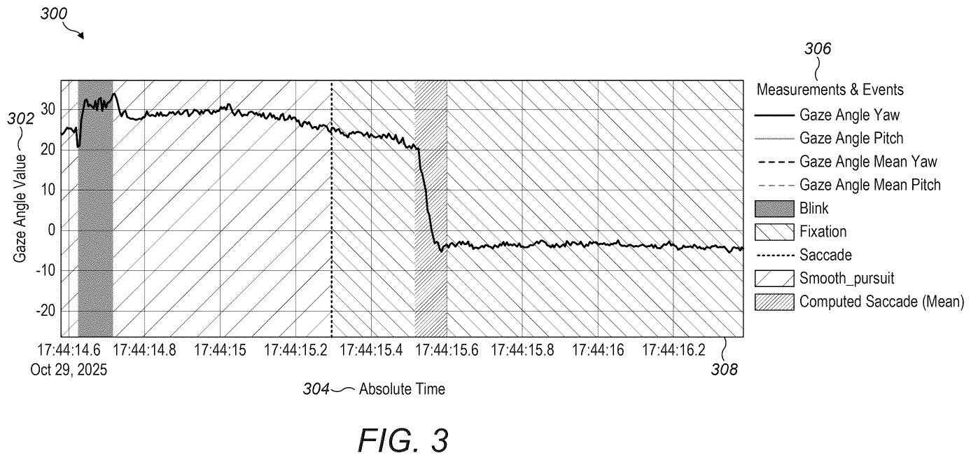

FIG. 2 and FIG. 3 show examples of the data collected from Participant 6 during the third trial. Specifically, FIG. 2 shows a plot 200 of synchronized sensor data with event markets over absolute time of approximately 23 seconds on the x-axis 204. The y-axis 202 shows the gaze angle value in degrees. The plot 200 shows measurements and events 206, namely: (1) Gaze Angle Yaw; (2) Gaze Angle Pitch; (3) Gaze Angle Mean Yaw; (4) Gaze Angle Mean Pitch; (5) Blink; (6) Fixation; (7) Saccade; (8) Smooth_pursit; and (9) Computed Saccade (Mean).

FIG. 3 shows a plot 300 of synchronized sensor data with event markets over absolute time of approximately the first 2 seconds on the x-axis 304 that are shown in FIG. 2. The y-axis 302 shows the gaze angle value in degrees. The plot 300 shows measurements and events 306, namely: (1) Gaze Angle Yaw; (2) Gaze Angle Pitch; (3) Gaze Angle Mean Yaw; (4) Gaze Angle Mean Pitch; (5) Blink; (6) Fixation; (7) Saccade; (8) Smooth_pursit; and (9) Computed Saccade (Mean).

### i. Smooth Pursuit

Participants were instructed to maintain continuous foveal gaze on a stimulus moving at a constant angular velocity along the horizontal plane. This phase was designed to induce controlled Yaw variations in gaze. This metric serves as an indicator of the participant's ability to predict and match stimulus velocity. An example of this can be seen in the areas shaded as “smooth_pursuit” in FIG. 2.

### ii. Smooth Pursuit Derived Features

These metrics measure how well the eye follows a moving target. The script uses a Savgol Filter to create a “perfect” path and calculates the error (RMSE) between the real gaze and that path.

### iii. Saccadic Transitions

To evaluate ballistic eye movement control, the stimulus underwent “step” displacements. At pseudo-random intervals, the stimulus would instantaneously “jump” to a new coordinate on the screen. Participants were required to execute a rapid gaze shift to the new target location. An example of this can be seen in the areas shaded as “Computed Saccade (Mean)” in FIG. 3. The key metrics for these movements were related to measurement of saccadic peak velocity, amplitude, and duration.

### iv. Saccadic Features

Saccades are the fast jumps the eyes make. The script identifies these by looking for monotonic segments (continuous movement in one direction) and calculating the speed and distance.

### v. Static Fixation

Every saccadic transition was immediately followed by a stationary period. Participants were required to maintain a stable gaze on the target for a duration of about 2 seconds. This phase allows for the assessment of gaze stability and the presence of micro-drifts or unwanted intrusive saccades during intoxication. An example of this can be seen in the areas shaded as “fixation” in FIG. 2.

### b. Fixation Features

Fixations occur when the eye is trying to stay perfectly still. The script trims the first 0.5 s of every fixation (to avoid the “settling” movement) and then measures “shakiness.”

### c. Audio Measures

For the audio task, participants were asked to speak for about 1-2 minutes about a subject of their choosing.

### 2. Trial Progression and Ground Truth

The experimental protocol consisted of a series of repeated trials (Trials 1 through 4). The progression was designed to capture the physiological transition from a sober baseline to peak impairment.

Baseline (Trial 1): Conducted at a sober state to establish individual oculomotor norms.

Alcohol Administration: Following Trial 1, participants were administered an amount of alcohol calculated for their age, height and gender in such a way as not to reach a dangerous level. After each alcohol administration session followed a 30 minute break and then performing the tasks (about 20 minutes). This resulted in about a 50 minute gap between alcohol administration sessions.

Ground Truth Monitoring: At the conclusion of each trial, Breath Alcohol Concentration (BrAC) was recorded using a Breathalyzer and subjective fatigue was captured via the Karolinska Sleepiness Scale (KSS).

### 3. Data Acquisition

Data acquisition is shown in Table 2.

TABLE 2

| Device | Data | Sampling rate |
|---|---|---|
| iPad tablet | Video data RGB | >200 Hz |
| | | (depending on the |
| | | recording conditions) |
| Microphone & camera | Video with audio | — |

In this disclosure only the gaze data from the iPad recording will be discussed.

### C. Key Findings

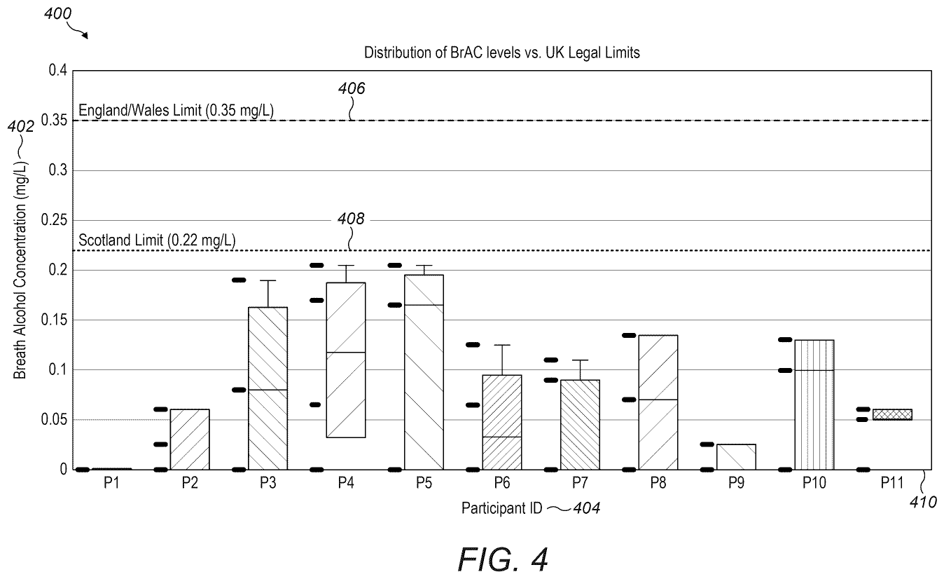

Turning to FIG. 4, shown is a plot 400 of the distribution of Breath Alcohol Concentration (BrAC) levels of the 10 participants in the study on the x-axis 404 against BrAC in mg/L on the y-axis 402. Also shown are the legal limits of Scotland (0.22 mg/L) 408 and England/Wales (0.35 mg/L) 406. Note the distribution of measured breath alcohol concentration. For all participants, at all times, the measurements were well below the England driving limit 406 and below the Scotland driving limit 408. This is important to keep in mind when interpreting the results especially when the changes in the features are small.

Linear Mixed-Effects Models (LMM) were used to provide a robust statistical foundation by separating global trends from individual participant baselines; these models are presented below for a few of the features that turned out to be significant.

### 1. Smooth Pursuit Gaze Angle Pitch (Mean_Rmse_Pitch) (Vertical) Error

The Linear Mixed-Effects Model reveals a clear hierarchy of influence. Breath Alcohol Concentration (BrAC) is the only highly significant predictor of Smooth Pursuit Gaze Angle Pitch (Mean_rmse_pitch) (vertical) gaze error (p=0.001). Even when controlled for the passage of time (Trial Number) and the participant's subjective level of tiredness (KSS), the chemical presence of alcohol remains the primary driver of oculomotor degradation.

Vertical Precision Loss: For every 1.0 mg/L increase in BrAC, the Smooth Pursuit Gaze Angle Pitch (Mean_rmse_pitch) (vertical) tracking error (RMSE Pitch) increases by 0.34 degrees.

Independence from Fatigue: While fatigue (KSS) showed a marginal trend (p=0.053), it did not reach the threshold of significance. This is a critical finding: it suggests that change in Smooth Pursuit Gaze Angle Pitch (Mean_rmse_pitch) (vertical) a “shaky” vertical gaze is a specific signature of alcohol impairment, rather than just a byproduct of being tired or bored with the task.

Consistency Across Individuals: The low Group Variance (0.012) indicates that while individuals have different “sober baselines,” their physiological reaction to alcohol follows a remarkably similar trajectory.

Dependent Variable: Smooth Pursuit Gaze Angle Pitch (Mean_rmse_pitch) (vertical) Mean_rmse_pitch|Observations: 281|Groups: 10.

Further data is shown in Table 3.

TABLE 3

| Predictor | Coefficient (β) | Std. Error | z-value | p-value | [95% Conf. Interval] |
|---|---|---|---|---|---|
| Intercept | 0.367 | 0.039 | 9.459 | <0.001 | [0.291, 0.443] |
| brac_mg_l | 0.340 | 0.098 | 3.467 | 0.001 | [0.148, 0.532] |
| trial_num | −0.005 | 0.006 | −0.809 | 0.418 | [−0.016, 0.007] |
| kss_score | 0.013 | 0.007 | 1.936 | 0.053 | [−0.000, 0.026] |

Random Effects are shown in Table 4.

TABLE 4

| Group Variable | Variance | Std. Error |
|---|---|---|
| Group (Intercept) | 0.012 | 0.102 |
| Residual Scale | 0.0032 | — |

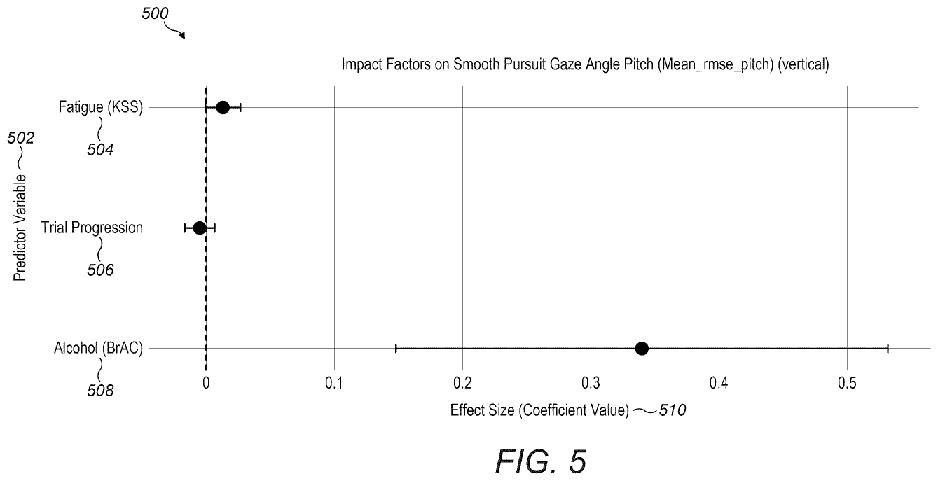

Turning to FIG. 5, shown is a plot 500 of Impact Factors on Smooth Pursuit Gaze Angle Pitch (Mean_rmse_pitch) (vertical). The x-axis 510 shows effect size (coefficient value) and the y-axis 502 shows predictive value divided into Fatigue (KSS) 504, Trial Progression 506, and Alcohol (BrAC) 508.

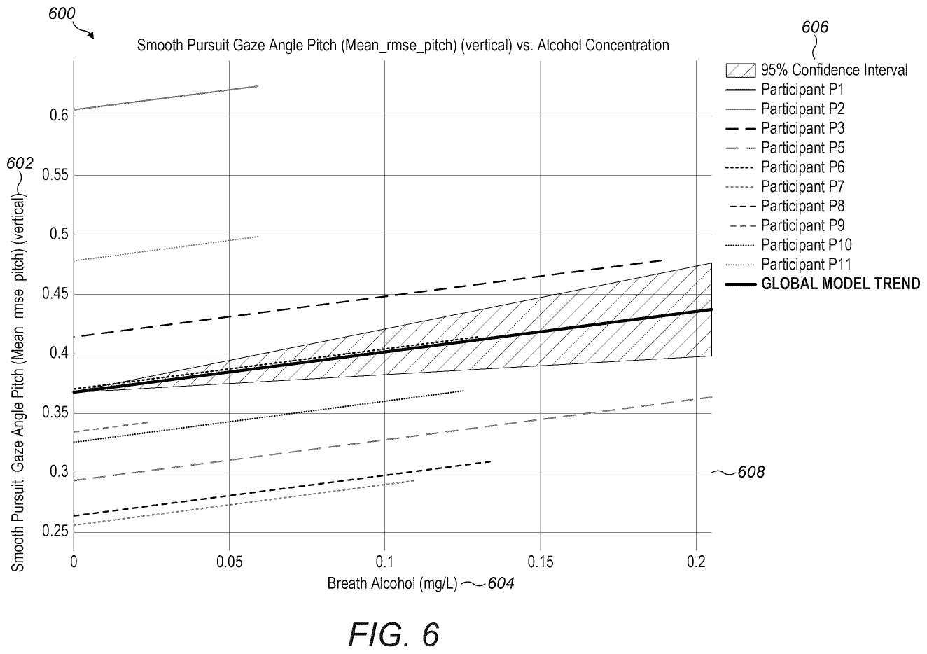

Turning to FIG. 6, shown is a plot 600 of Smooth Pursuit Gaze Angle Pitch (Mean_rmse_pitch) (vertical) vs. Alcohol Concentration. The x-axis 604 is BrAC in mg/L. The y-axis 602 is Smooth Pursuit Gaze Angle Pitch (Mean_rmse_pitch) (vertical) in degrees. The graph 608 shows data 606 for 10 participants along with a 95% confidence interval and a global model trend.

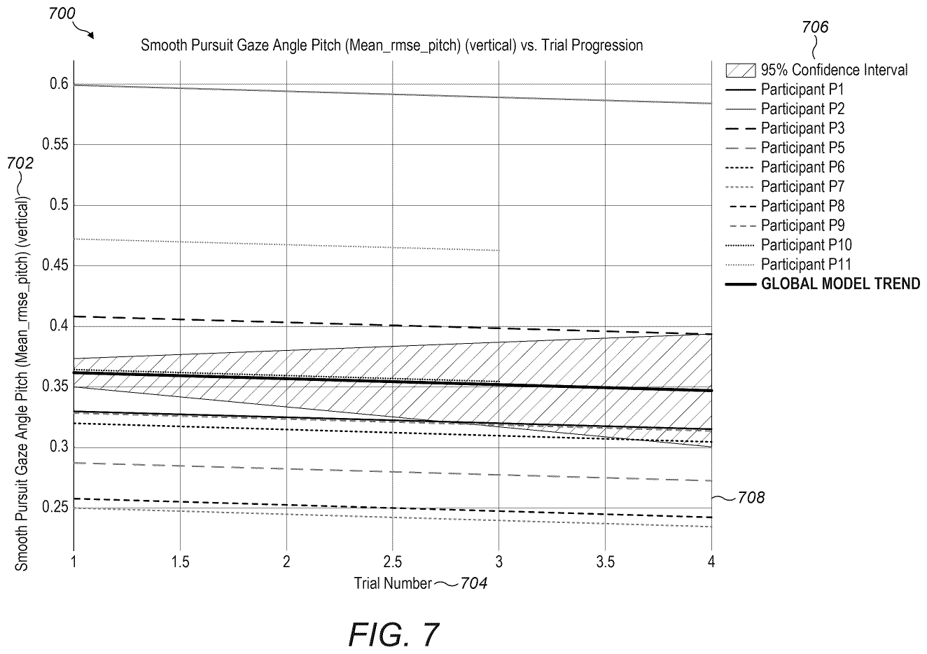

Turning to FIG. 7, shown is a plot 700 of Smooth Pursuit Gaze Angle Pitch (Mean_rmse_pitch) (vertical) vs. Trial Progression. The x-axis 704 is the trial number. The y-axis 702 is Smooth Pursuit Gaze Angle Pitch (Mean_rmse_pitch) (vertical) in degrees. The graph 708 shows data 706 for 10 participants along with a 95% confidence interval and a global model trend.

### 2. Smooth Pursuit Gaze Angle Mean Yaw (Mean_Rmse_Yaw) (Horizontal) Error

The Smooth Pursuit Gaze Angle Mean Yaw (Mean_rmse_yaw) (horizontal) results contrast sharply with the Smooth Pursuit Gaze Angle Pitch (Mean_rmse_pitch) (vertical) findings. While Smooth Pursuit Gaze Angle Pitch (Mean_rmse_pitch) (vertical) tracking showed a highly significant degradation linked to alcohol (p=0.001), the Smooth Pursuit Gaze Angle Mean Yaw (Mean_rmse_yaw) (horizontal) tracking error (Mean_rmse_yaw) did not (p=0.564).

Neither the Trial Number (p=0.249) nor the Fatigue Score (p=0.527) reached significance. This confirms that the Smooth Pursuit Gaze Angle Mean Yaw (Mean_rmse_yaw) (horizontal) system is not only robust against alcohol at these levels but also relatively resistant to “time-on-task” fatigue and repetition during the experiment. Further data is shown in Table 5.

TABLE 5

| | | Std. | | P-value | 95% Conf. |
|---|---|---|---|---|---|
| Predictor | Coefficient | Error | z-value | [P > \|z\|] | Interval |
| Intercept | 0.387 | 0.033 | 11.633 | <0.001* | [0.322, 0.452] |
| (Baseline) | | | | | |
| BrAC | 0.057 | 0.100 | 0.577 | 0.564 | [−0.138, 0.252] |
| (Alcohol) | | | | | |
| Trial | 0.007 | 0.006 | 1.153 | 0.249 | [−0.005, 0.018] |
| (Time) | | | | | |
| KSS | 0.004 | 0.007 | 0.633 | 0.527 | [−0.009, 0.018] |
| (Fatigue) | | | | | |

Random Effects are shown in Table 6.

TABLE 6

| Group Variable | Variance | Std. Error |
|---|---|---|
| Group (Intercept) | 0.008 | 0.063 |
| Residual Scale | 0.0035 | — |

The following three features below had more of an effect from trial progression. While this is a proxy for alcohol intake, it is also a proxy for fatigue/boredom or getting used to the task.

### 3. Saccade Mean_Peak_Speed_Deg_Per_Second Dynamics

A linear mixed-effects model was used to examine the impact of breath alcohol concentration (BrAC), trial progression, and subjective fatigue (KSS) on Saccade Mean_peak_speed_deg per second peak velocity. Results indicated that trial progression was a highly significant positive predictor of Saccade Mean_peak_speed_deg_per_second (B=121.68, SE=21.38, p<0.001), suggesting eyes became faster/ballistic as the session progressed. Conversely, BrAC was a significant negative predictor (B=−933.51, SE=365.74, p=0.011), indicating alcohol has a suppressant effect on Saccade Mean_peak_speed_deg_per second motor velocity. Fatigue also significantly reduced Saccade Mean_peak_speed_deg_per_second peak speed (B=−54.70, SE=24.05, p=0.023).

Further data is shown in Table 7.

TABLE 7

| Predictor | Coefficient | Std. Error | P-value | [95% Conf. Interval] | Interpretation |
|---|---|---|---|---|---|
| Intercept | 708.84 | 100.29 | <0.001 | [512.28, 905.4] | |
| BrAC (Alcohol) | −933.51 | 365.74 | 0.011 | [−1650.34, −216.67] | Alcohol slows ballistic movement |
| Trial (Time) | +121.68 | 21.38 | <0.001 | [79.77, 163.58] | Speed increases over session |
| KSS (Fatigue) | −54.70 | 24.05 | 0.023 | [−101.84, −7.55] | Tiredness reduces peak velocity |

Random Effects are shown in Table 8.

TABLE 8

| Group Variable | Variance | Std. Error |
|---|---|---|
| Group (Intercept) | 55959.76 | 136.93 |
| Residual Scale | 42419.62 | — |

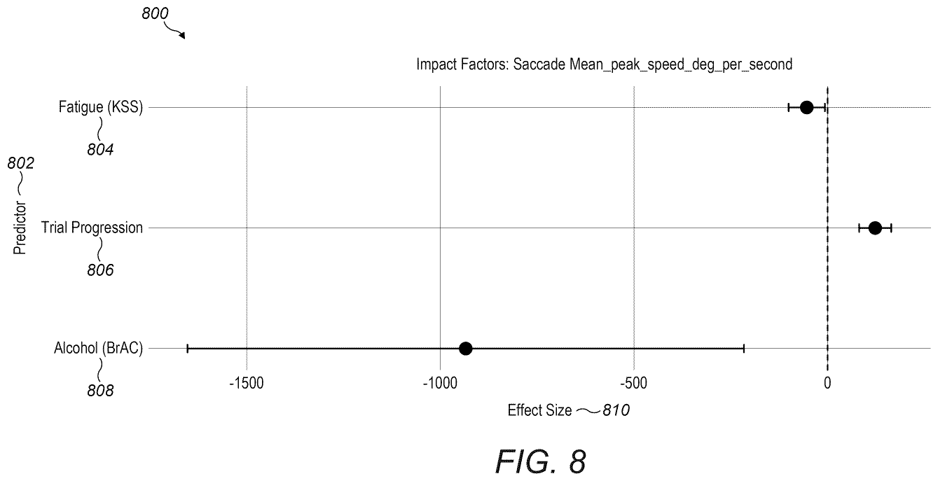

Turning to FIG. 8, shown is a plot 800 of Impact Factors on Saccade Mean_peak_speed_deg_per_second. The x-axis 810 shows effect size (coefficient value) and the y-axis 802 shows a predictor divided into Fatigue (KSS) 804, Trial Progression 806, and Alcohol (BrAC) 808.

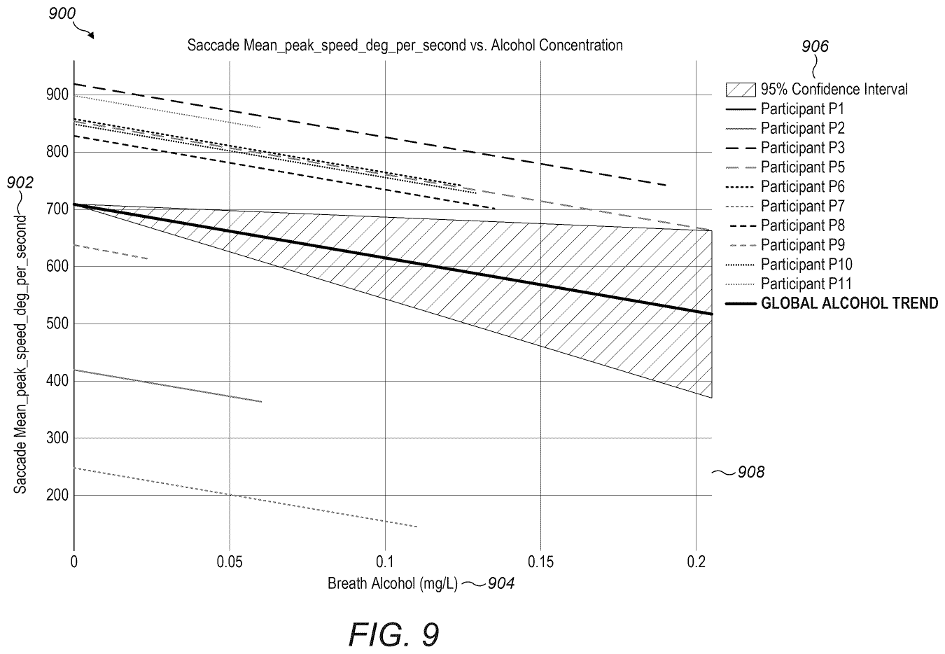

Turning to FIG. 9, shown is a plot 900 of Saccade Mean_peak_speed_deg_per_second vs. Alcohol Concentration. The x-axis 904 is BrAC in mg/L. The y-axis 902 is Smooth Gaze Feature 8 in degrees per second. The graph 908 shows data 906 for 10 participants along with a 95% confidence interval and a global model trend. This demonstrates statistically significant decline in saccadic motor speed as alcohol concentration increases (B=−933.51, p=0.011). While the model shows that fatigue (KSS score) also significantly slows saccades (B=−54.70, p=0.023), the magnitude of the alcohol effect is substantially larger. The graph demonstrates that alcohol's impact on peak speed is a dominant and independent factor, distinct from tiredness. The very high Group Variance (55959.76) indicates that participants have vastly different “baseline” saccade speeds (some have naturally “fast” eyes, others “slow”). By showing the “global model trend” alongside individual data, FIG. 9 demonstrates that despite these individual differences in baseline, the downward slope caused by alcohol is a consistent trend across the population.

### 4. Fixation Raw_Std_Dev_Yaw (Horizontal)—Fixation Stability

Fixation stability in the horizontal plane was assessed via the standard deviation of gaze yaw. The model revealed a significant negative relationship with trial progression (B=−0.778, SE=0.182, p<0.001) and Fixation Raw_std_dev_yaw (horizontal), supporting the hypothesis that the gaze becomes more rigid over time. Alcohol (B=6.336, SE=3.056, p=0.038) and fatigue (B=0.418, SE=0.210, p=0.046) both significantly increased Fixation Raw_std_dev_yaw (horizontal) instability, counteracting the compensatory stiffening effect of the trial progression.

Further data is shown in Table 9.

TABLE 9

| Predictor | Coefficient | Std. Error | P-value | [95% Conf. Interval] | Interpretation |
|---|---|---|---|---|---|
| Intercept | 2.135 | 0.958 | 0.026 | [0.256, 4.014] | |
| BrAC (Alcohol) | +6.336 | 3.056 | 0.038 | [0.347, 12.325] | Alcohol increases horizontal shakiness |
| Trial (Time) | −0.778 | 0.182 | <0.001 | [−1.135, −0.42] | Horizontal eye fixation becomes |
| | | | | | more stable over time |
| KSS (Fatigue) | +0.418 | 0.210 | 0.046 | [0.007, 0.829] | Fatigue increases horizontal drift |

Random Effects are shown in Table 10.

TABLE 10

| Group Variable | Variance | Std. Error |
|---|---|---|
| Group (Intercept) | 5.975 | 1.705 |
| Residual Scale | 3.0349 | — |

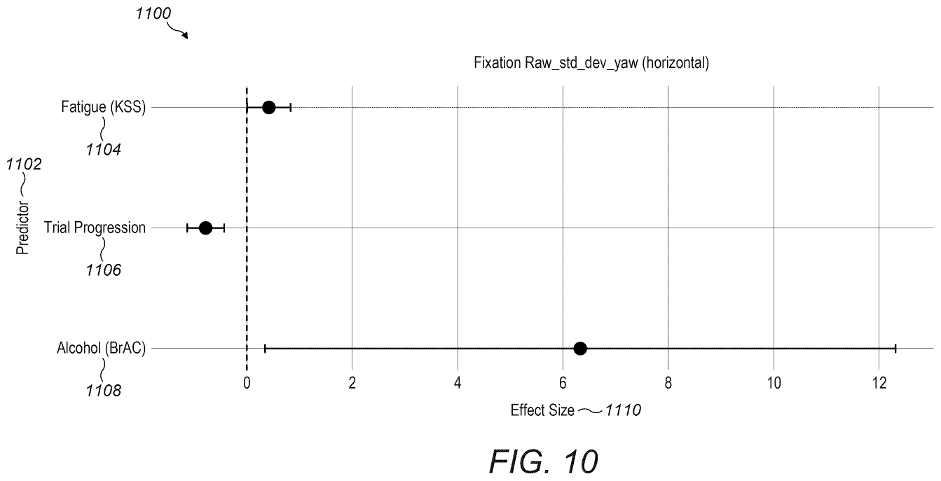

Turning to FIG. 10, shown is a plot 1100 of Impact Factors: Fixation Raw_std_dev_yaw (horizontal). The x-axis 1110 shows effect size and the y-axis 1102 shows a predictor divided into Fatigue (KSS) 1104, Trial Progression 1106, and Alcohol (BrAC) 1108.

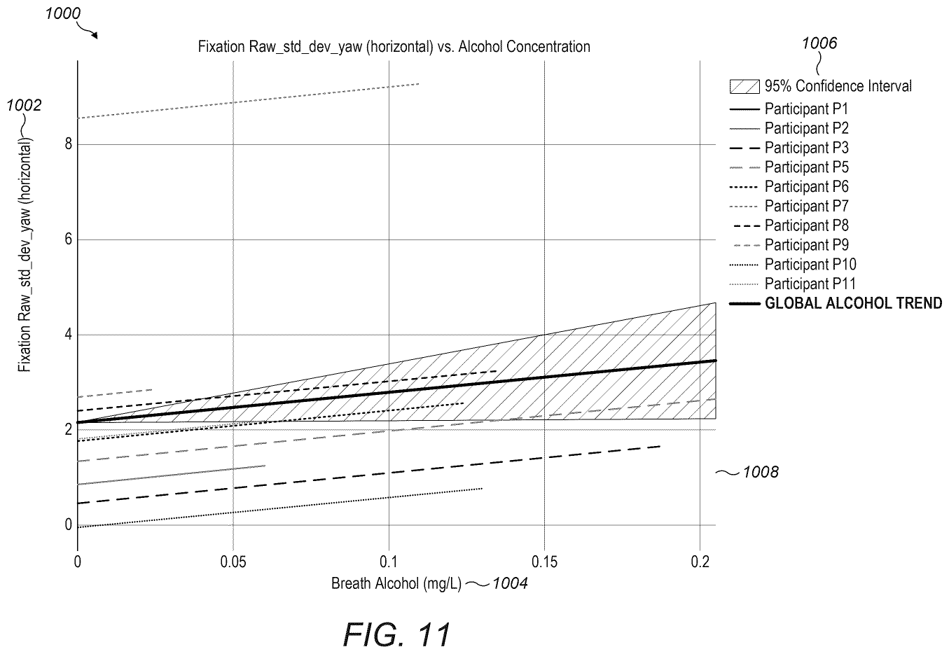

Turning to FIG. 11, shown is a plot 1000 of Fixation Raw_std_dev_yaw (horizontal) vs. Alcohol Concentration. The x-axis 1004 is BrAC in mg/L. The y-axis 1004 is Smooth Gaze Feature 3 in degrees. The graph 1008 shows data 1006 for 10 participants along with a 95% confidence interval and a global model trend. This demonstrates a statistically significant increase in horizontal gaze instability as breath alcohol concentration rises (B=6.336, p=0.038). The positive coefficient (6.336) demonstrates that as alcohol concentration increases, the participant's ability to maintain a steady horizontal gaze decreases. Visually, this is represented by the upward sloping “Global Alcohol Trend” line, indicating that the eyes become “shakier” (higher standard deviation of yaw) under the influence of alcohol. Subjective sleepiness also contributes to gaze shakiness, though to a lesser extent than alcohol. Interestingly, the negative coefficient for trial_num (−0.778) suggests a “learning effect” or stabilization over time, which the model has successfully accounted for to isolate the alcohol effect. The Group Variance (5.975) is quite high relative to the intercept, which is clearly visible in the graph by the widely different “starting points” (y-intercepts) for participants like P7 (very high instability) versus P10 (very low instability). This demonstrates the necessity of the Mixed-Effects Model; despite these different personal baselines, the upward slope (the alcohol effect) remains consistent across the group.

### 5. Fixation Mean_Std_Dev_Pitch (Vertical)—Fixation Stability

Fixation Mean_std_dev_pitch (vertical) fixation stability showed a significant decrease in standard deviation over the course of the experiment (B=−0.113, SE=0.042, p=0.007). However, unlike Fixation Raw_std_dev_yaw (horizontal) stability, Fixation Mean_std_dev_pitch (vertical) stability was not significantly impacted by alcohol (p=0.219) or subjective fatigue (p=0.225), suggesting Fixation Mean_std_dev_pitch (vertical) gaze locking is a more robust compensatory mechanism resistant to low-level pharmacological interference.

Further data is shown in Table 11.

TABLE 11

| Predictor | Coefficient | Std. Error | P-value | [95% Conf. Interval] | Interpretation |
|---|---|---|---|---|---|
| Intercept | 0.964 | 0.149 | <0.001 | [0.671, 1.257] | |
| BrAC (Alcohol) | 0.900 | 0.732 | 0.219 | [−0.534, 2.334] | No direct alcohol effect |
| Trial (Time) | −0.113 | 0.042 | 0.007 | [−0.194, −0.031] | Vertical eye fixation becomes more stable |
| | | | | | over time |
| KSS (Fatigue) | 0.051 | 0.042 | 0.225 | [−0.032, 0.135] | No fatigue effect |

Random Effects are shown in Table 12.

TABLE 12

| Group Variable | Variance | Std. Error |
|---|---|---|
| Group (Intercept) | 0.07 | 0.09 |
| Residual Scale | 0.1826 | — |

### 6. Summary of Results Table

Significant results are shown in Table 13.

TABLE 13

| | Alcohol | Trial | Fatigue | |
|---|---|---|---|---|
| | Effect | Effect | Effect | |
| Task & Metric | (BrAC) | (Time) | (KSS) | Conclusion |
| Smooth Pursuit Gaze Angle Pitch | Significant | Not | Marginal | Highly sensitive |
| (Mean_rmse_pitch) (vertical) | | Significant | | and independent |
| | | | | of fatigue. |
| Saccade | Significant | Significant | Significant | Mixed Effect. |
| Mean_peak_speed_deg_per_second | (Negative) | (Positive) | (Negative) | Alcohol and |
| | | | | fatigue slow |
| | | | | eyes, but |
| | | | | practice (Trial) |
| | | | | speeds them up. |
| Fixation Raw_std_dev_yaw | Significant | Significant | Significant | Passage of time |
| (horizontal) | | | | has a Gaze |
| | | | | “lock” effect, |
| | | | | while alcohol |
| | | | | and fatigue |
| | | | | increase |
| | | | | shakiness. |

### D. Audio Features

The audio analysis utilized Linear Mixed-Effects Models (Metric~BrAC+KSS+Trial) to isolate the specific chemical effect of alcohol from circadian fatigue and task habituation.

### 1. Audio Mfcc_Mean_20 (Mel-Frequency Cepstral Coefficients)

Audio mfcc_mean_20 emerged as a highly significant biomarker of intoxication, demonstrating a strong positive relationship with alcohol concentration (B=11.763, p<0.01). Similar to Smooth Pursuit Gaze Angle Pitch (Mean_rmse_pitch) (vertical) gaze precision, this metric remained independent of fatigue levels (p=0.652).

The random effects show that participants shared a very similar sober baseline.

Further data is shown in Table 14.

TABLE 14

| Predictor | Coefficient | Std. Error | P-value | [95% Conf. Interval] | Interpretation |
|---|---|---|---|---|---|
| Intercept | −0.640 | 0.651 | 0.3253 | [−1.916, 0.635] | |
| BrAC (Alcohol) | 11.763 | 3.356 | 0.0005 | [5.186, 18.341] | Significant effect from alcohol |
| Trial (Time) | −0.604 | 0.181 | 10.0008 | [−0.958, −0.25] | Effect on alcohol, which is expected |
| KSS (Fatigue) | 0.073 | 0.163 | 0.6516 | [−0.245, 0.392] | No fatigue effect |

Random effects are shown in Table 15.

TABLE 15

| Group Variable | Variance | Std. Error |
|---|---|---|
| Group (Intercept) | ~0 | N/A |
| Residual Scale | 0.463 | N/A |

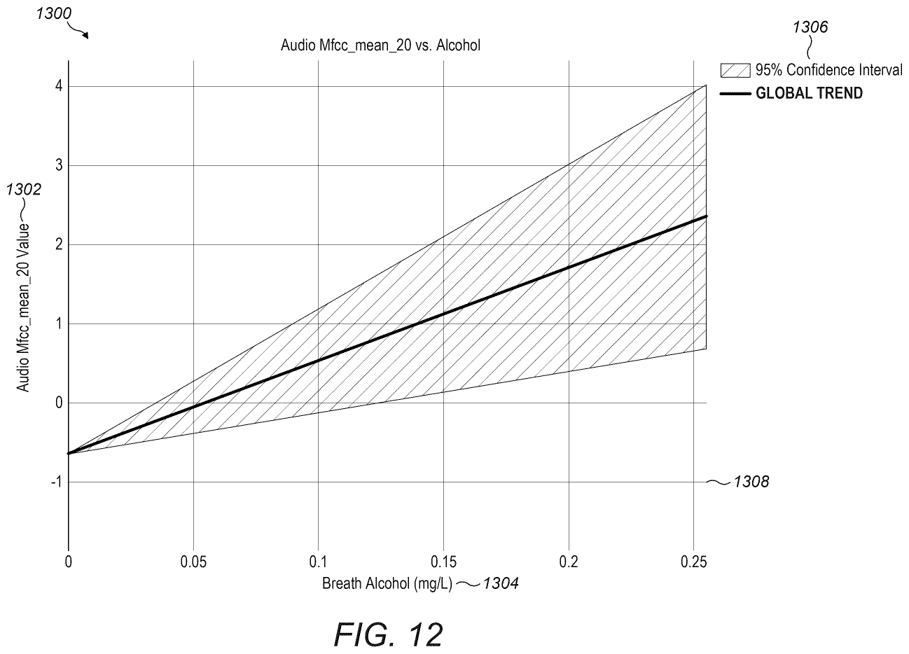

Turning to FIG. 12, shown is a plot 1300 of Audio mfcc_mean_20 vs. Alcohol. The x-axis 1304 is BrAC in mg/L. The y-axis 1302 is Audio mfcc_mean_20 Value in dB (Relative). The graph 1308 shows data 1306 a 95% confidence interval and a global trend. This demonstrates a highly significant positive correlation between alcohol concentration and high-frequency spectral detail (B=11.763, p=0.0005). Unlike some other features, this acoustic signature is completely independent of subjective fatigue (p=0.652), making it a robust biomarker for pure chemical intoxication. The Group Variance of ~0 shows that participants share a very similar baseline for this feature, leading to a highly reliable global alcohol trend across the cohort.

### 2. Audio Spectral_Rolloff_Mean (Mel-Frequency Cepstral Coefficients)

Audio spectral_rolloff_mean Spectral Rolloff Mean (the frequency below which 85% of spectral energy resides) was a significant biomarker of intoxication. Much like Smooth Pursuit Gaze Angle Pitch (Mean_rmse_pitch) (vertical) oculomotor tracking, this metric was not significantly impacted by subjective fatigue (p=0.769) or trial progression (p=0.071).

The random effects show that participants shared a very similar sober baseline.

Further data is shown in Table 16.

TABLE 16

| Predictor | Coefficient | Std. Error | P-value | [95% Conf. Interval] | Interpretation |
|---|---|---|---|---|---|
| Intercept | 2498.47 | 136.2549 | <0.01 | [2231.41, 2765.52] | |
| BrAC (Alcohol) | 1171.5 | 427.44 | <0.01 | [333.74, 2009.28] | Significant effect from alcohol |
| Trial (Time) | −44.56 | 24.67 | 0.07 | [−92.92, 3.8] | No trial progression effect |
| KSS (Fatigue) | 10.62 | 36.22 | 0.76 | [−60.37, 81.63] | No fatigue effect |

Random effects are shown in Table 17.

TABLE 17

| Group Variable | Variance | Std. Error |
|---|---|---|
| Group (Intercept) | ~0 | N/A |
| Residual Scale | 6374.56 | N/A |

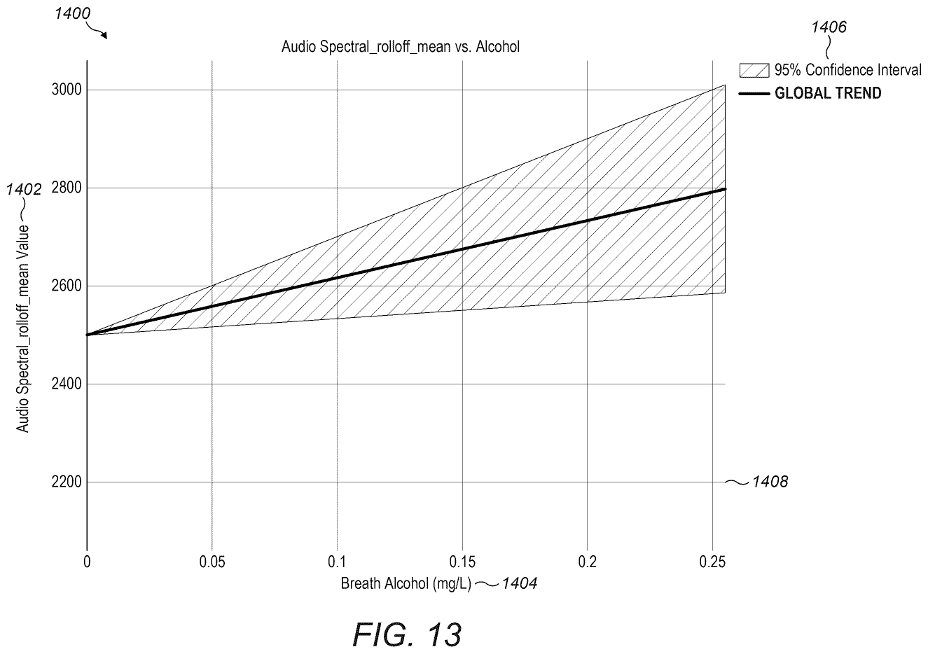

Turning to FIG. 13, shown is a plot 1400 of Audio spectral_rolloff mean vs. Alcohol. The x-axis 1404 is BrAC in mg/L. The y-axis 1402 is Audio spectral_rolloff_mean Value in Hz. The graph 1408 shows data 1406 a 95% confidence interval and a global trend. This demonstrate that alcohol consumption causes a significant upward shift in the spectral energy distribution of the voice (B=1171.51, p<0.01). Subjective tiredness (KSS) had almost no impact on spectral rolloff, proving that this change is a result of chemical intoxication rather than being sleepy. While there was a slight downward trend over time (likely due to vocal fatigue or habituation), it did not reach statistical significance, leaving alcohol as the primary driver. The Random Effects data shows a Group Variance of ~0, shows that participants share a very similar baseline for this feature.

### 3. Audio Spectral_Centroid_Mean

Audio spectral_centroid_mean (the “center of mass” of the vocal spectrum) showed a robust positive correlation with alcohol intake (B=576.047, p=0.0074). Consistent with Fixation Mean_std_dev_pitch (vertical) gaze stability findings, this metric was resistant to both trial progression (p=0.123) and subjective fatigue (p=0.603).

The random effects show that participants shared a very similar sober baseline.

Further data is shown in Table 18.

TABLE 18

| Predictor | Coefficient | Std. Error | P-value | [95% Conf. Interval] | Interpretation |
|---|---|---|---|---|---|
| Intercept | 1332.24 | 72.03 | <0.01 | [1191.06, 1473.42] | |
| BrAC (Alcohol) | 576.04 | 215.21 | <0.01 | [154.24, 997.85] | Significant effect from alcohol |
| Trial (Time) | −19.24 | 12.49 | 0.12 | [−43.73, 5.23] | No trial progression effect |
| KSS (Fatigue) | 9.89 | 19.04 | 0.6 | [−27.43, 47.23] | No fatigue effect |

Random effects are shown in Table 19.

TABLE 19

| Group Variable | Variance | Std. Error |
|---|---|---|
| Group (Intercept) | ~0 | N/A |
| Residual Scale | 1600 | N/A |

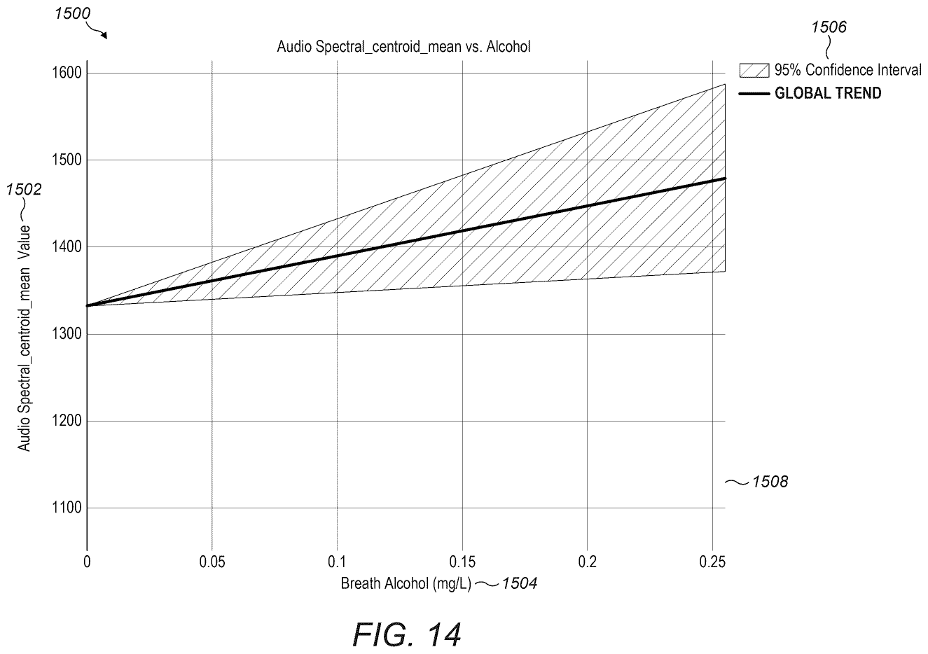

Turning to FIG. 14, shown is a plot 1500 of Spectral_rolloff_mean vs. Alcohol. The x-axis 1504 is BrAC in mg/L. The y-axis 1502 is Spectral_rolloff mean Value in Hz. The graph 1508 shows data 1506 a 95% confidence interval and a global trend. This demonstrates a statistically significant increase in the spectral center of mass of the voice as alcohol concentration increases (B=576.05, p=0.0074). Subjective fatigue showed no significant impact, proving that the change is not merely a result of being tired. Trial Progression also lacked significance, ensuring the effect is not due to vocal strain over the course of the experiment. The Random Effects data shows a Group Variance of ~0, shows that participants share a very similar baseline for this feature.

### E. Observations

Regarding the Smooth Pursuit Gaze Angle Pitch (Mean_rmse_pitch) (vertical) metric, while the changes relative to time progression (a proxy for alcohol intake and time passage) were not consistent across participants, some subsets of participants did exhibit similar trends.

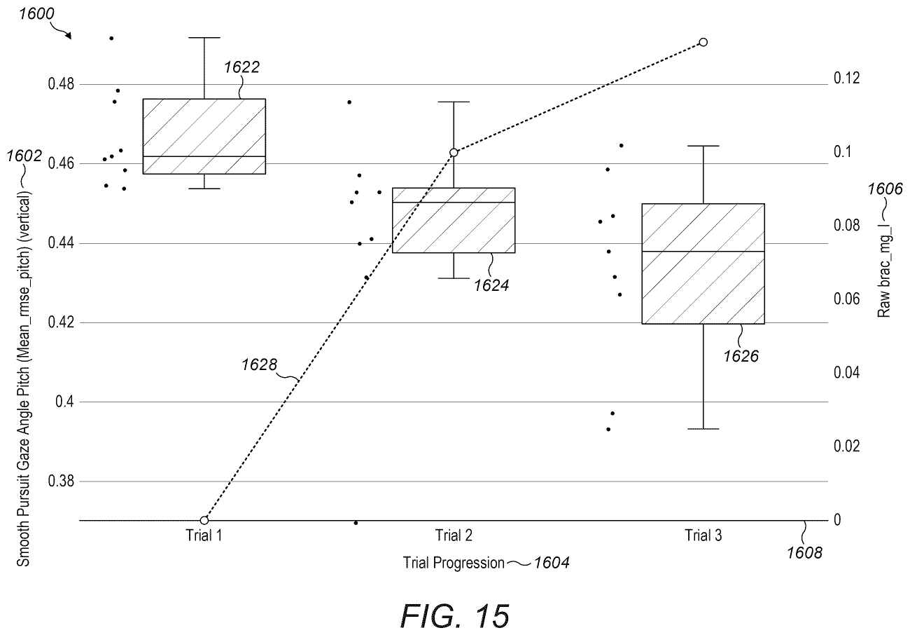

Turning to FIG. 15, shown is a plot 1600 showing Smooth Pursuit Gaze Angle Pitch (Mean_rmse_pitch) (vertical) and raw BrAC across 3 trials for participant 10. The x-axis 1604 shows the trial progression. The left y-axis 1602 shows Smooth Pursuit Gaze Angle Pitch (Mean_rmse_pitch) (vertical) degrees and is associated with the boxes 1622 1624 1626 in the graph 1608. The right y-axis 1606 shows raw BrAC in mg/L and is associated with what the dashed line 1628 in the graph 1608.

In the case of participant 10, whose data is presented in FIG. 15, a downward trend of Smooth Pursuit Gaze Angle Pitch (Mean_rmse_pitch) (vertical) is observed while the breath alcohol levels increase (dashed line 1628). This trend can be observed in 3 out of 10 participants.

### F. Further Applications

Further applications of the foregoing may include the following.
Increasing Sample Size: With N=10, the longitudinal nature of the data (multiple trials per participant) provides high statistical power for within-subject change. The results may not be further generalized to the broader population with larger sample sizes.
Increased Intoxication: All data points were collected at low intoxication levels. Further applications may include monitoring gaze control with increased impairment over the driving limit. The aim of such a system may be to output a binary outcome of below the limit vs over the limit.
Increasing Confounding Variables: While fatigue was monitored via KSS, further granularity in time beyond “Trial Progression” may provide a further proxy for alcohol absorption, metabolic changes, and task-induced boredom or learning effects. There may be linear relationships at different levels of alcohol intoxication and fatigue.
More Accurate Precision: The use of camera based gaze estimation might be influenced by many factors (glasses, lighting conditions, frame rate). Subtle changes in fixation stability may be partially influenced by the sensor's sampling conditions. Further, given the high sensitivity of the Smooth Pursuit Gaze Angle Pitch (Mean_rmse_pitch) (vertical) plane, the task paradigm may be expanded to include more complex vertical stimuli in that direction (e.g., vertical step-ramps or circular pursuit).

### G. Conclusions

This study demonstrates that high-fidelity oculomotor tracking may detect measurable functional changes in gaze control at breath alcohol concentrations (BrAC) significantly below UK legal driving limits. The most robust finding is the selective sensitivity of Smooth Pursuit Gaze Angle Pitch (Mean_rmse_pitch) (vertical) tracking system. Smooth Pursuit Gaze Angle Pitch (Mean_rmse_pitch) (vertical) error emerged as a primary biomarker, showing a highly significant correlation with alcohol intake (p=0.001) that remained independent of subjective fatigue.

In contrast, Smooth Pursuit Gaze Angle Mean Yaw (Mean_rmse_yaw) (horizontal) tracking proved resilient at these dosages, suggesting that low-level intoxication specifically impacts Smooth Pursuit Gaze Angle Pitch (Mean_rmse_pitch) (vertical) targets vertical gaze integration centers.

### CONCLUSION

In the foregoing specification, specific embodiments have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings.

Moreover, in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art. The term “coupled” as used herein is defined as connected, although not necessarily directly and not necessarily mechanically. A device or structure that is “configured” in a certain way is configured in at least that way but may also be configured in ways that are not listed.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

We claim:

1. A method comprising:
performing an intoxication test on a driver by a detection system in a car, comprising:
the driver commanding the detection system to start the car;
the detection system analyzing eye movements of the driver in the car to produce internal driver eye data;
the detection system performing the intoxication test of the driver based on the internal driver eye data;
wherein the analyzing eye movements comprises analyzing smooth pursuit gaze angle pitch of the eye movements.

2. The method of claim 1, wherein the analyzing smooth pursuit gaze angle pitch of the eye movements comprises using an intended path via Savitzky-Golay filtering techniques.

3. The method of claim 2, wherein the analyzing smooth pursuit gaze angle pitch of the eye movements further comprises using root mean squared error.

4. The method of claim 3, wherein the detection system further comprises:
analyzing the eye movements of the driver outside the car to produce external driver eye data; and
performing the intoxication test of the driver based on the internal driver eye data and the external driver eye data.

5. The method of claim 4, wherein if the detection system determines the driver is intoxicated, the detection system stops the driver from controlling the car.

6. A method comprising:
performing an intoxication test on a driver by a detection system in a car, comprising:
the driver commanding the detection system to start the car;
the detection system analyzing eye movements of the driver in the car to produce internal driver eye data;
the detection system performing the intoxication test of the driver based on the internal driver eye data;
wherein the analyzing eye movements comprises analyzing saccadic peak velocity of the eye movements.

7. The method of claim 6, wherein the analyzing saccadic peak velocity of the eye movements comprises calculating a standard deviation of horizontal raw yaw angle while changing gaze fixation from one location to another.

8. The method of claim 7, wherein the detection system further comprises:
analyzing the eye movements of the driver outside the car to produce external driver eye data; and
performing the intoxication test of the driver based on the internal driver eye data and the external eye data.

9. The method of claim 8, wherein if the detection system determines the driver is intoxicated, the detection system stops the driver from controlling the car.

10. A method comprising:
performing an intoxication test on a driver by a detection system in a car, comprising:
the driver commanding the detection system to start the car;
the detection system analyzing eye movements of the driver in the car to produce internal driver eye data;
the detection system performing the intoxication test of the driver based on the internal driver eye data;
wherein the analyzing eye movements comprises analyzing horizontal fixation stability of the eye movements.

11. The method of claim 10, wherein the analyzing horizontal fixation stability of the eye movements comprises calculating a standard deviation of horizontal raw yaw angle while viewing a static target.

12. The method of claim 11, wherein the detection system further comprises:
analyzing the eye movements of the driver outside the car to produce external driver eye data; and
performing the intoxication test of the driver based on the internal driver eye data and the external eye data.

13. The method of claim 12, wherein if the detection system determines the driver is intoxicated, the detection system stops the driver from controlling the car.

14. A method comprising:
performing an intoxication test on a driver by a detection system in a car, comprising:
the driver commanding the detection system to start the car;
the detection system requesting the driver to recite a phrase;
the driver repeating the phrase thereby generating internal driver audio;
the detection system analyzing the internal driver audio to produce internal driver audio data;
the detection system performing the intoxication test of the driver based on the internal driver audio data;
wherein the analyzing the internal driver audio comprises using a linear mixed-effects models to isolate specific chemical effect of alcohol on the driver.

15. The method as in claim 14, wherein the analyzing the internal driver audio further comprises using a plurality of coefficients of Mel-frequency cepstrum of the internal driver audio.

16. The method as in claim 14, wherein the analyzing the internal driver audio further comprises using a frequency below which a specified percentage of total spectral energy of the internal driver audio resides.

17. The method as in claim 14, wherein the analyzing the internal driver audio further comprises using an amplitude-weighted mean of frequencies present in the internal driver audio.

18. The method as in claim 14, further comprising:
the detection system analyzing eye movements of the driver to produce internal driver eye data;
the detection system performing the intoxication test of the driver based on the internal driver eye data;
wherein the analyzing eye movements comprises analyzing at least one of: (a) smooth pursuit gaze angle pitch; (b) saccadic peak velocity; and (c) horizontal fixation stability.
